(19) United States
(12) Patent Application Publication
(10) Pub. No.: US 2022/0313725 A1
(43) Pub. Date: Oct. 6, 2022

(54) Implantable Medical Device for the Delivery of a Nucleic Acid

(71) Applicant: Celanese EVA Performance Polymers LLC, Irving, TX (US)

(72) Inventors: Jeffrey C. Haley, Norwood, OH (US); Vijay Gyanani, Powell, OH (US); Brian D. Wilson, Grand Haven, MI (US); Harsh Patel, Union, KY (US); Narsi Devanathan, Union, KY (US)

(21) Appl. No.: 17/705,444

(22) Filed: Mar. 28, 2022

Related U.S. Application Data
(60) Provisional application No. 63/179,637, filed on Apr. 26, 2021.
(60) Provisional application No. 63/167,728, filed on Mar. 30, 2021.

(51) Int. Cl. A61K 31/7105; A61K 47/32; A61K 39/395; B29C 48/00; B29C 48/505

(52) U.S. Cl. CPC A61K 31/7105; A61K 47/32; A61K 39/3955; B29C 48/022; B29C 48/505; B29K 2105/0035

(57) ABSTRACT

An implantable medical device is provided. The device comprises a drug release layer, wherein the drug release layer comprises a naked nucleic acid dispersed within a polymer matrix. The polymer matrix includes an ethylene vinyl acetate copolymer and has a melting temperature of from about 20° C. to about 100° C. as determined in accordance with ASTM D3418-15 and a melt flow index of from about 0.2 to about 100 gram per 10 minutes as determined in accordance with ASTM D1238-20 at a temperature of 190° C. and a load of 2.16 kilograms.

### CROSS REFERENCE TO RELATED APPLICATIONS

The present application claims filing benefit of U.S. Provisional Patent Application Ser. No. 63/167,728 having a filing date of Mar. 30, 2021 and U.S. Provisional Patent Application Ser. No. 63/179,637 having a filing date of Apr. 26, 2021, which are incorporated herein by reference in their entirety.

### BACKGROUND OF THE INVENTION

Nucleic acids, such as mRNA and siRNA, have recently become a focal point for a substantial amount of gene therapy treatments, such as oncological treatments, vaccines, and so forth. For example, compared to DNA, ribonucleic acids (e.g., mRNA) are not stably integrated into the genome of the transfected cell, thus eliminating the concern that the introduced genetic material will disrupt the normal functioning of an essential gene. Extraneous promoter sequences are also not required for effective translation of the encoded protein, again avoiding possible deleterious side effects. One problem with ribonucleic acid-based gene therapy, however, is that it is far less stable than DNA, especially when it reaches the cytoplasm of a cell and is exposed to degrading enzymes. The presence of a hydroxyl group on the second carbon of the sugar moiety in mRNA, for example, causes steric hindrance that prevents the mRNA from forming the more stable double helix structure of DNA, and thus makes the mRNA more prone to hydrolytic degradation. In light of the above, ribonucleic acids are generally encapsulated into lipid particles (e.g., liposomes, solid lipid particles, etc.) to protect them from extracellular RNase degradation and simultaneously promote cellular uptake and endosomal escape. Unfortunately, however, problems nevertheless remain for their use in many applications. For example, it is difficult to controllably deliver nucleic acid-encapsulated lipid particles over a sustained period of time. One of the reasons for this difficulty is that the lipids employed in the particles tend to have a relatively low melting point, making it difficult to incorporate them into the processes and polymer materials used to form most conventional implantable medical devices.

As such, a need continues to exist for an implantable delivery device that is capable of delivering a nucleic acid over a sustained period of time.

### SUMMARY OF THE INVENTION

In accordance with one embodiment of the present invention, an implantable medical device is disclosed. The device comprises a drug release layer, wherein the drug release layer comprises a naked nucleic acid dispersed within a polymer matrix. The polymer matrix includes an ethylene vinyl acetate copolymer and has a melting temperature of from about 20° C. to about 100° C. as determined in accordance with ASTM D3418-15 and a melt flow index of from about 0.2 to about 100 gram per 10 minutes as determined in accordance with ASTM D1238-20 at a temperature of 190° C. and a load of 2.16 kilograms.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended drawings in which:

FIG. 1 is a perspective view of one embodiment of the implantable medical device of the present invention;

FIG. 2 is a cross-sectional view of the implantable medical device of FIG. 1;

FIG. 3 is a perspective view of another embodiment of the implantable medical device of the present invention; and

FIG. 4 is a cross-sectional view of the implantable medical device of FIG. 3.

Repeat use of references characters in the present specification and drawing is intended to represent same or analogous features or elements of the invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to an implantable medical device that is capable of delivering a nucleic acid to a patient (e.g., human, pet, farm animal, racehorse, etc.) over a sustained period of time to help prohibit and/or treat a condition, disease, and/or cosmetic state of the patient. The implantable medical device includes a “naked” nucleic acid dispersed within a polymer matrix, which includes one or more ethylene vinyl acetate copolymers. As used herein, a “naked” nucleic acid generally refers to a non-enveloped nucleic acid that lacks a surrounding carrier, such as a lipid, peptide, protein, carbohydrate (e.g., sugar), etc. The weight ratio of the polymer matrix to the nucleic acid is typically from about 1 to about 10, in some embodiments from about 1.1 to about 8, in some embodiments from about 1.2 to about 6, and in some embodiments, from about 1.5 to about 4. For example, in one embodiment, the implantable medical device may contain a drug release layer. The nucleic acid may constitute from about 1 wt. % to about 60 wt. %, in some embodiments from about 5 wt. % to about 50 wt. %, and in some embodiments, from about 10 wt. % to about 45 wt. % of the drug release layer, while the polymer matrix may constitute from about 40 wt. % to about 99 wt. %, in some embodiments from about 50 wt. % to about 95 wt. %, and in some embodiments, from about 55 wt. % to about 90 wt. % of the drug release layer. Notably, the ethylene vinyl acetate copolymer(s) employed within the polymer matrix are selected to have a certain melting temperature and melt flow index to help minimize the risk of nucleic acid degradation during processing. The ethylene vinyl acetate copolymer(s) and resulting polymer matrix may, for instance, have a melting temperature of from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. The melt flow index of the ethylene vinyl acetate copolymer(s) and the resulting polymer matrix may also be from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms.

Various embodiments of the present invention will now be described in more detail.

### I. Polymer Matrix

As indicated above, the polymer matrix contains at least ethylene vinyl acetate copolymer, which is generally derived from at least one ethylene monomer and at least one vinyl acetate monomer. The present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 55 wt. %, in some embodiments from about 30 wt. % to about 50 wt. %, in some embodiments from about 35 wt. % to about 48 wt. %, and in some embodiments, from about 38 wt. % to about 45 wt. % of the copolymer. Conversely, the ethylene content of the copolymer may likewise be within a range of from about 40 wt. % to about 80 wt. %, 45 wt. % to about 75 wt. %, in some embodiments from about 50 wt. % to about 80 wt. %, in some embodiments from about 52 wt. % to about 65 wt. %, and in some embodiments, from about 55 wt. % to about 62 wt. %. Among other things, such a comonomer content can help achieve a controllable, sustained release profile of the nucleic acid, while also still having a relatively low melting temperature that is more similar in nature to the melting temperature of the ethylene vinyl acetate copolymer(s). The density of the ethylene vinyl acetate copolymer may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55).

In certain embodiments, it may also be desirable to employ blends of an ethylene vinyl acetate copolymer and a hydrophobic polymer such as described below (e.g., ethylene vinyl acetate copolymer) such that the overall blend and polymer matrix have a melting temperature and/or melt flow index within the range noted above. For example, the polymer matrix may contain a first ethylene copolymer and a second ethylene copolymer having a melting temperature that is greater than the melting temperature of the first ethylene copolymer. The second ethylene copolymer may likewise have a melt flow index that is the same, lower, or higher than the corresponding melt flow index of the first ethylene copolymer. The first ethylene vinyl acetate copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second ethylene vinyl acetate copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first ethylene copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second ethylene copolymer may likewise constitute from about 20 wt. % to about 80 wt. %, in some embodiments from about 30 wt. % to about 70 wt. %, and in some embodiments, from about 40 wt. % to about 60 wt. % of the polymer matrix. Blends of an ethylene vinyl acetate copolymer and other hydrophobic polymers, such as described below, may also be employed within the polymer matrix.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain cases, ethylene vinyl acetate copolymer(s) constitute the entire polymer content of the polymer matrix. In other cases, however, it may be desired to include other polymers, such as other hydrophobic polymers and/or hydrophilic polymers, such as described below. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

If desired, the polymer matrix may also contain one or more plasticizers to help lower the processing temperature, thereby allowing higher melting point copolymers to be used without resulting in heat denaturation of the nucleic acid. Suitable plasticizers may include, for instance, fatty acids, fatty acids esters (e.g., triglycerides), fatty acid salts, fatty acid amides, organic phosphate esters, hydrocarbon waxes, etc., as well as mixtures thereof. The fatty acid may generally be any saturated or unsaturated acid having a carbon chain length of from about 8 to 22 carbon atoms, and in some embodiments, from about 10 to about 18 carbon atoms. If desired, the acid may be substituted. Suitable fatty acids may include, for instance, lauric acid, myristic acid, behenic acid, oleic acid, palmitic acid, stearic acid, ricinoleic acid, capric acid, neodecanoic acid, hydrogenated tallow fatty acid, hydroxy stearic acid, the fatty acids of hydrogenated castor oil, erucic acid, coconut oil fatty acid, etc., as well as mixtures thereof. Fatty acid derivatives may also be employed, such as fatty acid amides, such as oleamide, erucamide, stearamide, ethylene bis(stearamide), etc.; fatty acid salts (e.g., metal salts), such as calcium stearate, zinc stearate, magnesium stearate, iron stearate, manganese stearate, nickel stearate, cobalt stearate, etc.; fatty acid esters, such as fatty acid esters of aliphatic alcohols (e.g., 2-ethylhexanol, monoethylene glycol, isotridecanol, propylene glycol, pentraerythritol, etc.), fatty acid esters of glycerols (e.g., castor oil, sesame oil, etc.), fatty acid esters of polyphenols, sugar fatty acid esters, etc.; as well as mixtures of any of the foregoing. Hydrocarbon waxes, including paraffin waxes, polyolefin and oxidized polyolefin waxes, and microcrystalline waxes, may also be employed. Particularly suitable are acids, salts, or amides of stearic acid, such as stearic acid, calcium stearate, pentaerythritol tetrastearate, or N,N′-ethylene-bis-stearamide. When employed, the plasticizer(s) typically constitute from about 0.05 wt. % to about 1.5 wt. %, and in some embodiments, from about 0.1 wt. % to about 0.5 wt. % of the polymer matrix.

### II. Nucleic Acid

As indicated above, a naked nucleic acid is dispersed within the polymer matrix. As used herein, the term “nucleic acid” generally refers to a compound comprising a nucleobase and an acidic moiety, e.g., a nucleoside, nucleotide, polynucleotide, or a combination thereof. A “nucleoside” generally refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” generally refers to a nucleoside including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphdioester linkages, in which case the polynucleotides would comprise regions of nucleotides. For example, polynucleotides may contain three or more nucleotides are linear molecules, in which adjacent nucleotides are linked to each other via a phosphodiester linkage. The term “nucleic acid” also encompasses RNA as well as single and/or double-stranded DNA. More particularly nucleic acids may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-c-LNA having a 2′-amino functionalization), ethylene nucleic acids (ENA), cyclohexenyl nucleic acids (CeNA) or chimeras or combinations thereof.

Nucleic acids may be naturally occurring, for example, in the context of a genome, a transcript, a mRNA, tRNA, rRNA, siRNA, snRNA, plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Nucleic acids can be purified from natural sources, produced using recombinant expression systems and optionally purified, chemically synthesized, etc. The nucleic acids may also include nucleoside analogs, such as analogs having chemically modified bases or sugars, and backbone modifications. In some embodiments, the nucleic acid is or contains natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Modified nucleotide base pairing may be employed and encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In certain embodiments, the nucleic acid may be a polynucleotide (e.g., RNA polynucleotides, such as mRNA polynucleotides) in which one or more nucleobases has been modified for therapeutic purposes. In fact, in certain embodiments, a polynucleotide (e.g., RNA polynucleotide, such as mRNA polynucleotide) may be employed that includes a combination of at least two (e.g., 2, 3, 4 or more) of modified nucleobases. For example, suitable modified nucleobases in the polynucleotide may be a modified cytosine, such as 5-methylcytosine, 5-methyl-cytidine (m5C), N4-acetyl-cytidine (ac4C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine, etc.; modified uridine, such as 5-cyano uridine, 4′-thio uridine, pseudouridine (ψ), N1-methylpseudouridine (m1y), N1-ethylpseudouridine, 2-thiouridine (s2U), 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine (mo5U), 5-methoxyuridine, 2′-O-methyl uridine, etc.; modifined guanosine, such as α-thio-guanosine, inosine (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, etc.; modified adenine, such as α-thio-adenosine, 7-deaza-adenine, 1-methyl-adenosine (m1A), 2-methyl-adenine (m2A), N6-methyl-adenosine (m6A), 2,6-diaminopurine, etc.; as well as combinations thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the polynucleotide (e.g., RNA polynucleotide, such as mRNA polynucleotide) may be uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, polynucleotides function as messenger RNA (mRNA). “Messenger RNA” (mRNA) generally refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of a mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features that serve to overcome existing problems of effective polypeptide expression using nucleic-acid based therapeutics. The mRNA may contain at least one (one or more) ribonucleic acid (RNA) polynucleotide having an open reading frame encoding at least one polypeptide of interest. In some embodiments, a RNA polynucleotide of a mRNA encodes 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 polypeptides. In some embodiments, a RNA polynucleotide of a mRNA encodes at least 10, 20, 30, 40, 50, 60, 70, 80, 90 or 100 polypeptides. In some embodiments, a RNA polynucleotide of a mRNA encodes at least 100 or at least 200 polypeptides.

In some embodiments, the nucleic acids are therapeutic mRNAs. As used herein, the term “therapeutic mRNA” refers to a mRNA that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of various diseases and conditions, such as bacterial infections, viral infections, parasitic infections, cell proliferation disorders, genetic disorders, and autoimmune disorders. The mRNA may be designed to encode polypeptides of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

Particularly suitable therapeutic mRNAs are those that include at least one ribonucleic acid (RNA) polynucleotide having an open reading frame encoding at least one antigenic polypeptide, in which the RNA polynucleotide of the RNA includes at least one chemical modification. The chemical modification may, for instance, be pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine, and 2′-O-methyl uridine.

Although by no means required, the particular nature of the nucleic acid may also be selected to help improve its ability to be dispersed within the polymer matrix and delivered to a patient without significant degradation. For instance, it may be desired to co-deliver a conventional RNA (e.g., mRNA) with a self-amplifying RNA. Conventional mRNAs, for instance, generally include an open reading frame for the target antigen, flanked by untranslated regions and with a terminal poly(A) tail. After transfection, they drive transient antigen expression. Self-amplifying mRNAs, on the other hand, are capable of directing their self-replication, through synthesis of the RNA-dependent RNA polymerase complex, generating multiple copies of the antigen-encoding mRNA, and express high levels of the heterologous gene when they are introduced into the cytoplasm of host cells. Circular RNA (circRNA), which is a single-stranded RNA joined head to tail, may also be employed. The target RNA may be circularized, for example, by backsplicing of a non-mammalian exogenous intron or splint ligation of the 5′ and 3′ ends of a linear RNA. Examples of suitable circRNAs are described, for instance, in U.S. Patent Publication No. 2019/0345503, which is incorporated herein by reference thereto. Antisense RNA may also be employed, which generally has a base carried on a backbone subunit composed of morpholino backbone groups and in which the backbone groups are linked by inter-subunit linkages (both charged and uncharged) that allow the bases in the compound to hybridize to a target sequence in an RNA by Watson-Crick base pairing, thereby forming an RNA:oligonucleotide heteroduplex within the target sequence. Morpholino oligonucleotides with uncharged backbone linkages, including antisense oligonucleotides, are detailed, for example, in U.S. Pat. Nos. 5,698,685, 5,217,866, 5,142,047, 5,034,506, 5,166,315, 5,185, 444, 5,521,063, and 5,506,337, which are incorporated herein by reference. Other exemplary antisense oligonucleotides are described in U.S. Pat. Nos. 9,464,292, 10,131,910, 10,144,762, and 10,913,947, which are incorporated herein by reference.

In certain cases, the nucleic acid may be an aptamer, such as an RNA aptamer. An RNA aptamer may be any suitable RNA molecule that can be used on its own as a stand-alone molecule, or may be integrated as part of a larger RNA molecule having multiple functions, such as an RNA interference molecule. For example, an RNA aptamer may be located in an exposed region of an shRNA molecule (e.g., the loop region of the shRNA molecule) to allow the shRNA or miRNA molecule to bind a surface receptor on the target cell. After it is internalized, it may then be processed by the RNA interference pathways of the target cell. The nucleic acid that forms the nucleic acid aptamer may include naturally occurring nucleosides, modified nucleosides, naturally occurring nucleosides with hydrocarbon linkers (e.g., an alkylene), and/or or a polyether linker (e.g., a PEG linker) inserted between one or more nucleosides, modified nucleosides with hydrocarbon or PEG linkers inserted between one or more nucleosides, or a combination of thereof. In some embodiments, nucleotides or modified nucleotides of the nucleic acid aptamer can be replaced with a hydrocarbon linker or a polyether linker. Suitable aptamers may be described, for instance, in U.S. Pat. No. 9,464,293, which is incorporated herein by reference thereto.

Protein-fused nucleic acids may also be suitable for use in the present invention. For example, proteins (e.g., antibodies) may be covalently linked to RNA (e.g., mRNA). Such RNA-protein fusions may be synthesized by in vitro or in situ translation of mRNA pools containing a peptide acceptor attached to their 3′ ends. In one embodiment, after readthrough of the open reading frame of the message, the ribosome pauses when it reaches the designed pause site, and the acceptor moiety occupies the ribosomal A site and accepts the nascent peptide chain from the peptidyl-tRNA in the P site to generate the RNA-protein fusion. The covalent link between the protein and the RNA (in the form of an amide bond between the 3′ end of the mRNA and the C-terminus of the protein that it encodes) allows the genetic information in the protein to be recovered and amplified (e.g., by PCR) following selection by reverse transcription of the RNA. Once the fusion is generated, selection or enrichment is carried out based on the properties of the mRNA-protein fusion, or, alternatively, reverse transcription may be carried out using the mRNA template while it is attached to the protein to avoid the impact of the single-stranded RNA on the selection. Examples of such protein-fused nucleic acids are described, for instance, in U.S. Pat. No. 6,518,018, which is incorporated herein by reference. Ribozymes (e.g., DNAzyme and/or RNAzyme) may also be employed that are conjugated to nucleic acids having a sequence that catalytically cleaves RNA, such as described in U.S. Pat. No. 10,155,946, which is incorporated herein by reference.

Apart from single strand nucleic acids such as described above, various specific types of double strand nucleic acids may also be employed to help improve stability. Circular DNA (cDNA) and plasmid nucleic acids (e.g., pDNA), which are a closed circular form of DNA, may be employed in certain embodiments. Examples of such nucleic acids are described, for instance, in WO 2004/060277 which is incorporated herein by reference. Long double stranded DNA may also be employed. For instance, a scaffolded DNA origami may be employed in which the long single-stranded DNA is folded into a certain shape by annealing the scaffold in the presence of shorter oligonucleotides (“staples”) containing segments or regions of complementary sequences to the scaffold. Examples of such structures are described, for instance, in U.S. Patent Publication Nos. 2019/0142882 and 2018/0171386, which are incorporated herein by reference.

### II. Excipients

The drug release layer may also optionally contain one or more excipients, such as cell permeability enhancers (e.g., fatty acids, such as oleic acid), ribonucleic acid degradation inhibitors (e.g., RNAase and/or DNAse inhibitors), radiocontrast agents, hydrophilic compounds, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the drug release layer. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

Cell permeability enhancers may also be employed to help aid in delivery of the nucleic acid. Examples of such permeability enhancers may include, for instance, tight junction modifiers, cyclodextrin, trihydroxy salts (e.g., bile salts, such as sodium glycocholate or sodium fusidate), surfactants (e.g., sodium lauryl sulfate, sodium dodecyl sulfate, cetyltrimethyl ammonium bromide, lauryl betaine, polyoxyethylene sorbitan monopalmitate, etc.), saponin, fusidic acids and derivatives thereof, fatty acids and derivatives thereof (e.g., oleic acid, monoolein, sodium caprate, sodium laurate, etc.), pyrrolidones (e.g., 2-pyrrolidone), alcohols (e.g., ethanol), glycols (e.g., propylene glycol), azones (e.g., laurocapram), terpenes, chelating agents (e.g., EDTA), dendrimers, oxazolidines, diooxolanes (e.g., 2-n-nonyl-1,3-dioxolane), lipids (e.g., phospholipids), and so forth.

To help minimize the risk of nucleic acid degradation, a ribonucleic acid inhibitor may be employed. Representative inhibitors for this purpose may include, for instance, anti-nuclease antibodies and/or non-antibody inhibitors. Suitable nuclease antibodies may be anti-ribonuclease antibodies or anti-deoxyribonuclease antibodies. The anti-ribonuclease antibodies may be antibodies that inhibit one or more of the following ribonucleases or deoxyribonucleases: RNase A, RNase B, RNase C, RNase 1, RNase T1, micrococcal nuclease, S1 nuclease, mammalian ribonuclease 1 family, ribonuclease 2 family, mammalian angiogenins, RNase H family, RNase L, eosinophil RNase, messenger RNA ribonucleases (5′-3′ Exoribonucleases, 3′-5′ Exoribonucleases), decapping enzymes, deadenylases, E. coli endoribonucleases (RNase P, RNase III, RNase E, RNase I,I*, RNase HI, RNase HII, RNase M, RNase R, RNase IV, F; RNase P2,O, PIV, PC, RNase N), E. coli exoribonucleases (RNase II, PNPase, RNase D, RNase BN, RNase T, RNase PH, OligoRNase, RNase R), RNase Sa, RNase F1, RNase U2, RNase Ms, RNase St, DNase 1, S1 nuclease, and micrococcal nuclease. Suitable non-antibody nuclease inhibitors may likewise include, but are not limited to, diethyl pyrocarbonate, ethanol, formamide, guanidinium thiocyanate, vanadyl-ribonucleoside complexes, macaloid, sodium dodecylsulfate (SDS), proteinase K, heparin, hydroxylamine-oxygen-cupric ion, bentonite, ammonium sulfate, dithiothreitol (DTT), β-mercaptoethanol, cysteine, dithioerythritol, urea, polyamines (spermidine, spermine), detergents (e.g., sodium dodecylsulfate), tris (2-carboxyethyl) phosphene hydrochloride (TCEP), and so forth. Chelating agents are also suitable non-antibody nuclease inhibitors as such compounds can help bind cations (e.g., calcium, iron, etc.) that would otherwise cause degradation. The chelating agent may include, for instance, aminocarboxylic acids (e.g., ethylenediaminetetraacetic acid) and salts thereof, hydroxycarboxylic acids (e.g., citric acid, tartaric acid, ascorbic acid, etc.) and salts thereof, polyphosphoric acids (e.g., tripolyphosphoric acid, hexametaphosphoric acid, etc.) and salts thereof, and so forth. Desirably, the chelating agent is multidentate in that it is capable of forming multiple coordination bonds with metal ions to reduce the likelihood that any of the free metal ions. In one embodiment, for example, a multidentate chelating agent containing two or more aminodiacetic (sometimes referred to as iminodiacetic) acid groups or salts thereof may be utilized. One example of such a chelating agent is ethylenediaminetetraacetic acid (EDTA). Examples of suitable EDTA salts include calcium disodium EDTA, diammonium EDTA, disodium and dipotassium EDTA, trisodium and tripotassium EDTA, tetrasodium and tetrapotassium EDTA. Still other examples of similar aminodiacetic acid chelating agents include, but are not limited to, butylenediaminetetraacetic acid, (1,2-cyclohexylenediaminetetraacetic acid (CyDTA), diethylenetriaminepentaacetic acid (DTPA), ethylenediaminetetrapropionic acid, (hydroxyethyl)ethylenediaminetriacetic acid (HEDTA), triethanolamine EDTA, triethylenetetraminehexaacetic acid (TTNA), 1,3-diamino-2-hydroxypropane-N,N,N′,N′-tetraacetic acid (DHPTA), methyliminodiacetic acid, propylenediaminetetraacetic acid, ethylenediiminodipropanedioic acid (EDDM), 2,2′-bis(carboxymethyl)iminodiacetic acid (ISA), ethylenediiminodibutandioic acid (EDDS), and so forth. Still other suitable multidentate chelating agents include N,N,N′,N′-ethylenediaminetetra(methylenephosphonic)acid (EDTMP), nitrilotrimethyl phosphonic acid, 2-aminoethyl dihydrogen phosphate, 2,3-dicarboxypropane-1,1-diphosphonic acid, meso-oxybis(butandionic acid) (ODS), and so forth.

To help further control the release rate from the implantable medical device, for example, a hydrophilic compound may also be incorporated into the drug release layer that is soluble and/or swellable in water. When employed, the weight ratio of the ethylene vinyl acetate copolymer(s) the hydrophilic compounds within the drug release layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the drug release layer, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the drug release layer. Suitable hydrophilic compounds may include, for instance, polymers, non-polymeric materials, such as fatty acids or salts thereof (e.g., stearic acid, citric acid, myristic acid, palmitic acid, linoleic acid, etc., as well as salts thereof), biocompatible salts (e.g., sodium chloride, calcium chloride, sodium phosphate, etc.), hydroxy-functional compounds as described below, etc. Examples of suitable hydrophilic polymers include, for instance, sodium, potassium and calcium alginates, cellulosic compounds (e.g., hydroxymethylcellulose, carboxymethylcellulose, ethylcellulose, methylcellulose, etc.), agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

One or more nonionic, anionic, and/or amphoteric surfactants may also be employed to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the membrane layer. Nonionic surfactants, which typically have a hydrophobic base (e.g., long chain alkyl group or an alkylated aryl group) and a hydrophilic chain (e.g., chain containing ethoxy and/or propoxy moieties), are particularly suitable. Some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. Particularly suitable nonionic surfactants may include ethylene oxide condensates of fatty alcohols, polyoxyethylene ethers of fatty acids, polyoxyethylene sorbitan fatty acid esters, and sorbitan fatty acid esters, etc. The fatty components used to form such emulsifiers may be saturated or unsaturated, substituted or unsubstituted, and may contain from 6 to 22 carbon atoms, in some embodiments from 8 to 18 carbon atoms, and in some embodiments, from 12 to 14 carbon atoms. Sorbitan fatty acid esters (e.g., monoesters, diester, triesters, etc.) that have been modified with polyoxyethylene are one particularly useful group of nonionic surfactants. These materials are typically prepared through the addition of ethylene oxide to a 1,4-sorbitan ester. The addition of polyoxyethylene converts the lipophilic sorbitan ester surfactant to a hydrophilic surfactant that is generally soluble or dispersible in water. Such materials are commercially available under the designation TWEEN® (e.g., TWEEN® 80, or polyethylene (20) sorbitan monooleate).

### III. Device Configuration

A. Drug Release Layer

As noted above, a drug release layer may be formed from the polymer matrix, nucleic acid, and optional excipients. The drug release layer and/or implantable medical device may have a variety of different geometric shapes, such as cylindrical (rod), disc, ring, doughnut, helical, elliptical, triangular, ovular, etc. In one embodiment, for example, the drug release layer and/or implantable medical device may have a generally circular cross-sectional shape so that the overall structure is in the form of a cylinder (rod) or disc. In such embodiments, the drug release layer and/or implantable medical device will typically have a diameter of from about 0.5 to about 50 millimeters, in some embodiments from about 1 to about 40 millimeters, and in some embodiments, from about 5 to about 30 millimeters. The length of the drug release layer and/or implantable medical device may vary, but is typically in the range of from about 1 to about 25 millimeters. Cylindrical devices may, for instance, have a length of from about 5 to about 50 millimeters, while disc-shaped devices may have a length of from about 0.5 to about 5 millimeters.

The drug release layer may be formed through a variety of known techniques, such as by hot-melt extrusion, injection molding, solvent casting, dip coating, spray coating, microextrusion, coacervation, etc. In one embodiment, a hot-melt extrusion technique may be employed. Hot-melt extrusion is generally a solvent-free process in which the components of the drug release layer (e.g., ethylene vinyl acetate copolymer(s), nucleic acid(s), optional excipients, etc.) may be melt blended and optionally shaped in a continuous manufacturing process to enable consistent output quality at high throughput rates. This technique is particularly well suited to ethylene vinyl acetate copolymers as they typically exhibit a relatively high degree of long-chain branching with a broad molecular weight distribution. This combination of traits can lead to shear thinning of the copolymer during the extrusion process, which help facilitates hot-melt extrusion. Furthermore, the polar viny acetate comonomer units can serve as an “internal” plasticizer by inhibiting crystallization of the polyethylene chain segments. This may lead to a lower melting point of the copolymer, which further enhances its ability to be processed with the nucleic acid.

During a hot-melt extrusion process, melt blending generally occurs at a temperature that is similar to or slightly above the melting temperature of the ethylene vinyl acetate copolymer(s). The melt blending temperature may, for example, be from about 30° C. to about 100° C., in some embodiments, from about 40° C. to about 80° C., and in some embodiments, from about 50° C. to about 70° C. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around the center of the screw. A feed section and melt section may be defined along the length of the screw. The feed section is the input portion of the barrel where the ethylene vinyl acetate copolymer(s) and/or nucleic acid are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be extruded through an orifice (e.g., die) and formed into pellets, sheets, fibers, filaments, etc., which may be thereafter shaped into a drug release layer using a variety of known shaping techniques, such as injection molding, compression molding (e.g., vacuum compression molding), nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present invention. In one embodiment, an injection molding apparatus may be employed that includes a first mold base and a second mold base, which together define a mold cavity having the shape of the drug release layer. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape for the drug release layer (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 50° C. to about 120° C., in some embodiments from about 60° C. to about 110° C., and in some embodiments, from about 70° C. to about 90° C.

As indicated above, another suitable technique for forming a drug release layer of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present invention. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

B. Membrane Layer

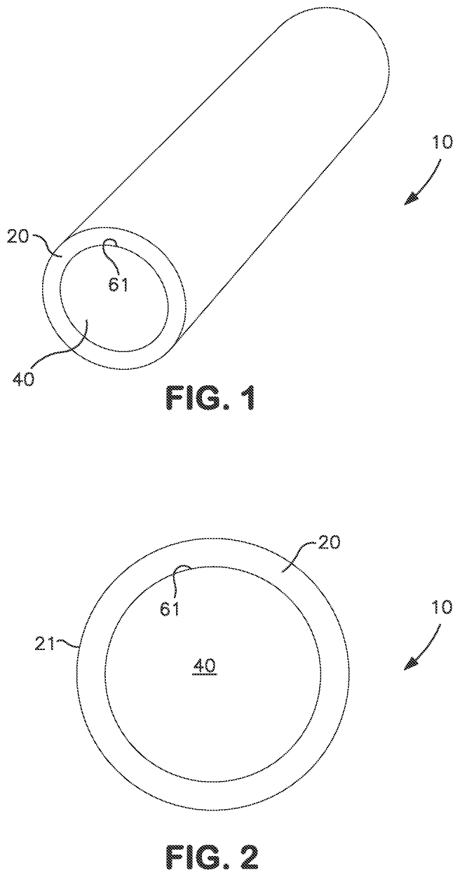

In some cases, the implantable medical device may be multilayered in that it contains at least one membrane layer positioned adjacent to an outer surface of the drug release layer (i.e., the “core”). The number of membrane layers may vary depending on the particular configuration of the device, the nature of the nucleic acid, and the desired release profile. For example, the device may contain only one membrane layer. Referring to FIGS. 1-2, for example, one embodiment of an implantable medical device 10 is shown that contains a core 40 having a generally circular cross-sectional shape and is elongated so that the resulting device is generally cylindrical in nature. The core 40 defines an outer circumferential surface 61 about which a membrane layer 20 is circumferentially disposed. Similar to the core 40, the membrane layer 20 also has a generally circular cross-sectional shape and is elongated so that it covers the entire length of the core 40. During use of the device 10, a nucleic acid is capable of being released from the core 40 and through the membrane layer 20 so that it exits from an external surface 21 of the device.

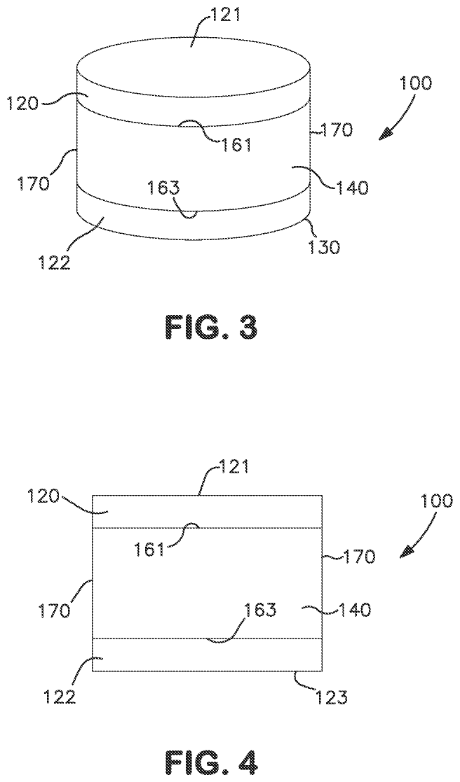

Of course, in other embodiments, the device may contain multiple membrane layers. In the device of FIGS. 1-2, for example, one or more additional membrane layers (not shown) may be disposed over the membrane layer 20 to help further control release of the nucleic acid. In other embodiments, the device may be configured so that the core is positioned or sandwiched between separate membrane layers. Referring to FIGS. 3-4, for example, one embodiment of an implantable medical device 100 is shown that contains a core 140 having a generally circular cross-sectional shape and is elongated so that the resulting device is generally disc-shaped in nature. The core 140 defines an upper outer surface 161 on which is positioned a first membrane layer 120 and a lower outer surface 163 on which is positioned a second membrane layer 122. Similar to the core 140, the first membrane layer 120 and the second membrane layer 122 also have a generally circular cross-sectional shape that generally covers the core 140. If desired, edges of the membrane layers 120 and 122 may also extend beyond the periphery of the core 140 so that they can be sealed together to cover any exposed areas of an external circumferential surface 170 of the core 140. During use of the device 100, a nucleic acid is capable of being released from the core 140 and through the first membrane layer 120 and second membrane layer 122 so that it exits from external surfaces 121 and 123 of the device. Of course, if desired, one or more additional membrane layers (not shown) may also be disposed over the first membrane layer 120 and/or second membrane layer 122 to help further control release of the nucleic acid.

Regardless of the particular configuration employed, the membrane layer(s) generally contain a membrane polymer matrix that contains a hydrophobic polymer. The membrane polymer matrix typically constitutes from about 30 wt. % to 100 wt. %, in some embodiments, from about 40 wt. % to about 99 wt. %, and in some embodiments, from about 50 wt. % to about 90 wt. % of a membrane layer. When employing multiple membrane layers, it is typically desired that each membrane layer contains a membrane polymer matrix that includes such a hydrophobic polymer. For example, a first membrane layer may contain a first membrane polymer matrix and a second membrane layer may contain a second membrane polymer matrix. In such embodiments, the first and second membrane polymer matrices each contain a hydrophobic polymer, which may be the same or different.

The polymer(s) used in the membrane polymer matrix are generally hydrophobic in nature so that they can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the membrane polymer matrix. In certain embodiments, the membrane polymer matrix may contain a semi-crystalline olefin copolymer. The melting temperature of such an olefin copolymer may, for instance, range from about 20° C. to about 100° C., in some embodiments from about 25° C. to about 80° C., in some embodiments from about 30° C. to about 70° C., in some embodiments from about 35° C. to about 65° C., and in some embodiments, from about 40° C. to about 60° C., such as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, the present inventors have discovered that certain aspects of the copolymer can be selectively controlled to help achieve the desired release properties. For instance, the polar monomeric content of the copolymer may be selectively controlled to be within a range of from about 20 wt. % to about 60 wt. %, in some embodiments from about 25 wt. % to about 55 wt. %, in some embodiments from about 30 wt. % to about 50 wt. %, in some embodiments from about 35 wt. % to about 48 wt. %, and in some embodiments, from about 38 wt. % to about 45 wt. % of the copolymer. Conversely, the olefin monomeric content of the copolymer may likewise be within a range of from about 40 wt. % to about 80 wt. %, 45 wt. % to about 75 wt. %, in some embodiments from about 50 wt. % to about 80 wt. %, in some embodiments from about 52 wt. % to about 65 wt. %, and in some embodiments, from about 55 wt. % to about 62 wt. %.

The hydrophobic polymer used in the membrane polymer matrix may also be the same or different than the ethylene vinyl acetate copolymer(s) employed in the drug release layer. In one embodiment, for instance, both the drug release layer (core) and the membrane layer(s) employ the same polymer (e.g., ethylene vinyl acetate copolymer). In yet other embodiments, the membrane layer(s) may employ a hydrophobic polymer (e.g., α-olefin copolymer) that has a lower melt flow index than the ethylene vinyl acetate copolymer employed in the drug release layer. Among other things, this can further help control the release of the nucleic acid from the device. For example, the ratio of the melt flow index of a ethylene vinyl acetate copolymer employed in the drug release layer to the melt flow index of a hydrophobic polymer employed in the membrane layer(s) may be from about 1 to about 20, in some embodiments about 2 to about 15, and in some embodiments, from about 4 to about 12. The melt flow index of the hydrophobic polymer in the membrane layer(s) may, for example, range from about 1 to about 80 g/10 min, in some embodiments from about 2 to about 70 g/10 min, and in some embodiments, from about 5 to about 60 g/10 min, as determined in accordance with ASTM D1238-13 at a temperature of 190° C. and a load of 2.16 kilograms. Examples of suitable ethylene vinyl acetate copolymers that may be employed include those available from Celanese under the designation ATEVA® (e.g., ATEVA® 4030AC or 2861A).

The membrane layer(s) used in the device may optionally contain a nucleic acid, such as described above, which are dispersed within the membrane polymer matrix. The nucleic acid in the membrane layer(s) may be the same or different than those employed in the core. Regardless, when a nucleic acid is employed in a membrane layer, it is generally desired that the membrane layer generally contains the nucleic acid in an amount such that the ratio of the concentration (wt. %) of the nucleic acid in the core to the concentration (wt. %) of the nucleic acid in the membrane layer is greater than 1, in some embodiments about 1.5 or more, and in some embodiments, from about 1.8 to about 4. When employed, nucleic acids typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of a nucleic acid prior to release from the drug release layer. When multiple membrane layers are employed, each membrane layer may generally contains the nucleic acid in an amount such that the ratio of the weight percentage of the nucleic acid in the drug release layer to the weight percentage of the nucleic acid in the membrane layer is greater than 1, in some embodiments about 1.5 or more, and in some embodiments, from about 1.8 to about 4.

The membrane layer(s) may also optionally contain one or more excipients as described above, such as radiocontrast agents, hydrophilic compounds, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

To help further control the release rate from the implantable medical device, for example, a hydrophilic compound may also be incorporated into the membrane layer such as described above. When employed, the weight ratio of the hydrophobic polymers to the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 50 wt. %, in some embodiments from about 2 wt. % to about 40 wt. %, and in some embodiments, from about 5 wt. % to about 30 wt. % of the membrane layer, while hydrophobic polymers typically constitute from about 50 wt. % to about 99 wt. %, in some embodiments from about 60 wt. % to about 98 wt. %, and in some embodiments, from about 70 wt. % to about 95 wt. % of the membrane layer.

In one particular embodiment, the membrane layer(s) may contain a hydrophilic compound that is in the form of a plurality of water-soluble particles distributed within a membrane polymer matrix. In such embodiments, the particle size of the water-soluble particles may be controlled to help achieve the desired delivery rate. More particularly, the median diameter (D50) of the particles may be about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. A variety of different materials may be employed to form such particles, such as fatty acids or salts thereof (e.g., stearic acid, citric acid, myristic acid, palmitic acid, linoleic acid, etc., as well as salts thereof), cellulosic compounds (e.g., hydroxymethylcellulose, carboxymethylcellulose, ethylcellulose, methylcellulose, etc.), biocompatible salts (e.g., sodium chloride, calcium chloride, sodium phosphate, etc.), hydroxy-functional compounds, and so forth. In particularly suitable embodiments, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present invention include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof.

One or more nonionic, anionic, and/or amphoteric surfactants may also be employed such as described above to help create a uniform dispersion. When employed, such surfactant(s) typically constitute from about 0.05 wt. % to about 8 wt. %, and in some embodiments, from about 0.1 wt. % to about 6 wt. %, and in some embodiments, from about 0.5 wt. % to about 3 wt. % of the membrane layer.

The membrane layer(s) may be formed using the same or a different technique than used to form the core, such as by hot-melt extrusion, injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the implantable device. As described above, the drug release and membrane layer(s) may be each individually formed by heating and compressing the respective polymer compression into the desired shape while under vacuum. Once formed, the drug release and membrane layer(s) may be stacked together to form a multi-layer precursor and thereafter and compression molded in the manner as described above to form the resulting implantable device.

### IV. Use of Device

Through selective control over the particular nature of the device and the manner in which it is formed, the resulting device can be effective for sustained release over a nucleic acid over a prolonged period of time. For example, the implantable medical device can release the nucleic acid for a time period of about 5 days or more, in some embodiments about 10 days or more, in some embodiments from about 20 days to about 60 days, and in some embodiments, from about 25 days to about 50 days (e.g., about 30 days). Further, the nucleic acid can be released in a controlled manner (e.g., zero order or near zero order) over the course of the release time period. After a time period of 15 days, for example, the cumulative release ratio of the implantable medical device may be from about 20% to about 70%, in some embodiments from about 30% to about 65%, and in some embodiments, from about 40% to about 60%. Likewise, after a time period of 30 days, the cumulative release ratio of the implantable medical device may still be from about 40% to about 85%, in some embodiments from about 50% to about 80%, and in some embodiments, from about 60% to about 80%. The “cumulative release ratio” may be determined by dividing the amount of the nucleic acid released at a particulate time interval by the total amount of nucleic acid initially present, and then multiplying this number by 100.

Of course, the actual dosage level of the nucleic acid delivered will vary depending on the particular nucleic acid employed and the time period for which it is intended to be released. The dosage level is generally high enough to provide a therapeutically effective amount of the nucleic acid to render a desired therapeutic outcome, i.e., a level or amount effective to reduce or alleviate symptoms of the condition for which it is administered. The exact amount necessary will vary, depending on the subject being treated, the age and general condition of the subject to which the nucleic acid is to be delivered, the capacity of the subject's immune system, the degree of effect desired, the severity of the condition being treated, the particular nucleic acid selected and mode of administration of the composition, among other factors. An appropriate effective amount can be readily determined by one of skill in the art. For example, an effective amount will typically range from about 5 μg to about 200 mg, in some embodiments from about 5 μg to about 100 mg per day, and in some embodiments, from about 10 μg to about 1 mg of the nucleic acid delivered per day.

The device may be implanted subcutaneously, orally, mucosally, etc., using standard techniques. The delivery route may be intrapulmonary, gastroenteral, subcutaneous, intramuscular, into the central nervous system (e.g., intrathecal), intraperitoneum, intraorgan, etc. In one embodiment, the implantable device may be particularly suitable for delivering a nucleic acid for cancer treatment. In such embodiments, the device may be placed in a tissue site of a patient in, on, adjacent to, or near a tumor, such as a tumor of the pancreas, biliary system, gallbladder, liver, small bowel, colon, brain, lung, eye, etc. The device may also be employed together with current systemic chemotherapy, external radiation, and/or surgery. The device may also be delivered intrathecally to treat and/or prohibit a variety of different conditions, such as cancer, neurological diseases (e.g., neurodegenerative disease, such as spinal muscular atrophy or amyotrophic lateral sclerosis), etc., and/or for use in pain management. In such embodiments, the device may be implanted into the spinal canal or directly into the intrathecal space (subarachnoid space), which is the space that holds the cerebrospinal fluid. For example, intrathecal administration may be accomplished by implanting the device into an Ommaya reservoir (a dome-shaped container that is placed under the scalp during surgery; it holds the drugs as they flow through a small tube into the brain) or directly into the cerebrospinal fluid in the lower part of the spinal column.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### Test Methods

The release of a nucleic acid (e.g., mRNA) may be determined using an in vitro method. More particularly, implantable device samples may be placed in 150 milliliters of an aqueous sodium azide solution. The solutions may be enclosed in UV-protected, 250-ml Duran® flasks. The flasks may then be placed into a temperature-controlled water bath and continuously shaken at 100 rpm. A temperature of 37° C. may be maintained through the release experiments to mimic in vivo conditions. Samples may be taken in regular time intervals by completely exchanging the aqueous sodium azide solution. The concentration of the nucleic acid in solution may be determined via UV/Vis absorption spectroscopy using a Cary 1 split beam instrument. From this data, the amount of the nucleic acid released per sampling interval (microgram per hour) may be calculated and plotted over time (hours). Further, the cumulative release ratio of the nucleic acid may also be calculated as a percentage by dividing the amount of the nucleic acid released at each sampling interval by the total amount of nucleic acid initially present, and then multiplying this number by 100. This percentage is then plotted over time (hours).

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An implantable medical device comprising a drug release layer, wherein the drug release layer comprises a naked nucleic acid dispersed within a polymer matrix, wherein the polymer matrix includes an ethylene vinyl acetate copolymer and has a melting temperature of from about 20° C. to about 100° C. as determined in accordance with ASTM D3418-15 and a melt flow index of from about 0.2 to about 100 gram per 10 minutes as determined in accordance with ASTM D1238-20 at a temperature of 190° C. and a load of 2.16 kilograms.

2. The implantable medical device of claim 1, wherein the weight ratio of the polymer matrix to the nucleic acid is from about 1 to about 10.

3. The implantable medical device of claim 1, wherein the ethylene vinyl acetate copolymer has a melting temperature of from about 20° C. to about 100° C. as determined in accordance with ASTM D3418-15.

4. The implantable medical device of claim 1, wherein ethylene vinyl acetate copolymers constitute the entire polymer content of the polymer matrix.

5. The implantable medical device of claim 1, wherein the polymer matrix further includes a plasticizer.

6. The implantable medical device of claim 1, wherein the polymer matrix further includes a hydrophobic polymer.

7. The implantable medical device of claim 6, wherein the polymer matrix includes a first ethylene vinyl acetate copolymer and a second ethylene vinyl acetate copolymer.

8. The implantable medical device of claim 1, wherein the vinyl acetate content of the copolymer is from about 10 wt. % to about 60 wt. %.

9. The implantable medical device of claim 1, wherein the ethylene vinyl acetate polymer has a melt flow index of from about 0.2 to about 100 grams per 10 minutes as determined in accordance with ASTM D1238-20 at a temperature of 190° C. and a load of 2.16 kilograms.

10. The implantable medical device of claim 1, wherein the nucleic acid includes a ribonucleic acid.

11. The implantable medical device of claim 10, wherein the ribonucleic acid includes mRNA.

12. The implantable medical device of claim 11, wherein the mRNA includes a therapeutic mRNA containing at least one ribonucleic acid polynucleotide having an open reading frame encoding at least one antigenic polypeptide.

13. The implantable medical device of claim 1, wherein the device has a generally circular cross-sectional shape.

14. The implantable medical device of claim 13, wherein the device has a diameter of from about 0.5 to about 50 millimeters.

15. The implantable medical device of claim 1, wherein the device is in the form of a cylinder.

16. The implantable medical device of claim 1, wherein the device is in the form of a disc.

17. The implantable medical device of claim 1, wherein the drug release layer further includes a ribonucleic acid degradation inhibitor.

18. The implantable medical device of claim 17, wherein the ribonucleic acid inhibitor includes an anti-ribonuclease antibody.

19. The implantable medical device of claim 17, wherein the ribonucleic acid inhibitor includes a chelating agent.

20. The implantable medical device of claim 1, wherein the drug release layer further includes a cell permeability enhancer.

21. The implantable medical device of claim 1, wherein the drug release layer further includes a hydrophilic compound.

22. The implantable medical device of claim 21, wherein the hydrophilic compound includes a hydrophilic polymer.

23. The implantable medical device of claim 22, wherein the hydrophilic polymer includes a sodium, potassium or calcium alginate, carboxymethylcellulose, agar, gelatin, polyvinyl alcohol, polyalkylene glycol, collagen, pectin, chitin, chitosan, poly-1-caprolactone, polyvinylpyrrolidone, poly(vinylpyrrolidone-co-vinyl acetate), polysaccharide, hydrophilic polyurethane, polyhydroxyacrylate, dextran, xanthan, hydroxypropyl cellulose, methylcellulose, protein, ethylene vinyl alcohol copolymer, water-soluble polysilane, water-soluble silicone, water-soluble polyurethane, or a combination thereof.

24. The implantable medical device of claim 1, wherein the nucleic acid constitutes from about 1 wt. % to about 60 wt. % of the drug release layer and the polymer matrix constitutes from about 40 wt. % to about 99 wt. % of the drug release layer.

25. The implantable medical device of claim 1, further comprising a membrane layer positioned adjacent to an outer surface of the drug release layer.

26. The implantable medical device of claim 25, wherein the membrane layer is free of a nucleic acid.

27. The implantable medical device of claim 25, wherein the membrane layer comprises a membrane polymer matrix comprising a hydrophobic polymer.

28. The implantable medical device of claim 27, wherein the hydrophobic polymer includes an ethylene vinyl acetate copolymer.

29. The implantable medical device of claim 27, wherein the membrane polymer matrix is formed entirely from hydrophobic polymers.

30. The implantable medical device of claim 27, wherein the membrane polymer matrix also contains a hydrophilic compound.

31. A method for forming the implantable medical device of claim 1, the method comprising melt blending the naked nucleic acid and the polymer matrix within an extruder.

32. The method of claim 31, wherein melt blending occurs at a temperature of from about 30° C. to about 100° C.

33. The method of claim 31, wherein the extruder includes a rotatable screw having a length and diameter, wherein the ratio of the length to the diameter is from about 10 to about 50.

34. A method for prohibiting and/or treating a condition, disease, and/or cosmetic state of a patient, the method comprising subcutaneously implanting the device of claim 1 in the patient.
